(19) United States
(12) Patent Application Publication
(10) Pub. No.: US 2005/0260260 A1
(43) Pub. Date: Nov. 24, 2005

(54) Liposome compositions for the delivery of macromolecules

(76) Inventors: Edward Kisak, La Jolla, CA (US); Bret Coldren, Raleigh, NC (US)

Correspondence Address: Edward Kisak, 226 Playa del Norte, La Jolla, CA, 92037, US

(21) Appl. No.: 11/132,368

(22) Filed: May 19, 2005

Related U.S. Application Data
(60) Provisional application No. 60/572,913, filed on May 19, 2004.

(51) Int. Cl. A61K 9/127

(52) U.S. Cl. 424/450

(57) ABSTRACT

This invention provides for a liposome composition which demonstrates greatly increased therapeutic efficacy when used to deliver encapsulated macromolecular drugs. The liposome composition excludes the use of sterols, sterol derivatives, and cationic lipids, contrary to conventional formulations. The invention liposome is also unique in that it utilizes low gel to fluid phase transition temperature lipids in its membrane.

### REFERENCES CITED

U.S. Patent Documents:

| | | |
|---|---|---|
| 5,213,804 | May 1993 | Martin et.al. |
| 5,468,499 | November 1995 | Chan et.al. |
| 5,814,335 | September 1998 | Webb et.al. |
| 6,083,923 | July 2000 | Hardee et.al. |
| 6,333,314 | December 2001 | Kasid et.al. |
| 6,534,484 | March 2003 | Wheeler et.al. |

Other Publications:
1.) Barron, Uyechi and Szoka “Cationic lipids are essential for gene delivery mediated by intravenous administration of lipoplexes” Human Gene Therapy, V.6, 1999.
2.) Bruckdorfer, et. al. “The effect of partial replacements of membrane cholesterol by other steroids on the osmotic fragility and glycerol permeability of erythrocytes” Biochim Biophys Acta. 1969 Jul. 15; 183(2):334-45
3.) Charrois, et. al. “Drug release rate influences the pharmacokinetics, biodistribution, therapeutic activity and toxicity of pegylated liposomal doxorubicin formulations in murine breast cancer” Biochimica et Biophysica Acta, 1663(1-2) 2004.
4.) Charrois and Allen “Multiple injections of pegylated liposomal Doxorubicin: pharmacokinetics and therapeutic activity” J Pharmacol Exp Ther. 306 (3): 1058-67.Epub 2004 Jun. 13.
5.) Choi et. al. “Gene delivery to the rat liver using cationic lipid emulsion/DNA complex:comparison between intra-arterial, intraportal and intravenous administration” Korean journal of radiology, 3(3), 2002.
6.) Chou et. al. “Effect of composition on the stability of liposomal irinotecan prepared by a pH gradient method” J BioSci and BioEng, 2003, 95(4)
7.) Grit and Crommelin “Chemical stability of liposomes: implications for their physical stability” Chem. Phys. Lipids 64, 1993.
8.) Irma, et. al. “Improved efficacy of ciprofloxacin administered in polyethylene glycol-coated liposomes for treatment of Klebsiella pneumoniae pneumonia in rats” Antimicrob Agenets and Chemo, May 2001.
9.) Lorna et. al “Biodistribution of long-circulating PEG-liposomes in a murine model of established subcutaneous abscesses” Biochimica et Biophysica Acta 1561 (2002)
10.) Ma, et. al. “Lipid-Mediated Delivery of Oligonucleotdies to Pulmonary Endothelium” Am. J. Respir. Cell Mol. Biol. Vol. 27, 2002
11.) Mahato et. al. “Physicochemical and pharmacokinetic characteristics of plasmid DNA/cationic liposome complexes” J. Pharm. Sci 84:1267-1271. 1995.
12.) Needham and Nunn “Elastic deformation and failure of lipid bilayer membranes containing cholesterol”. Biophys. J., 58, 997-1009
13.) Rosie, et.al. “Pharmacokinetics and tissue disposition in monkeys of an antisense oligonucleotide inhibitor Ha-Ras encapsulated in stealth liposomes”, Pharma Res V. 16, No. 8, 1999
14.) Zhu, et. al. “The effect of vincristine-polyanion complexes in Stealth liposomes on pharmacokinetics, toxicity and anti tumor activity” Cancer Chemother Pharmacol, (2000) 39.

### FIELD OF THE INVENTION

This invention relates to compositions for improving the efficacy of macromolecular drug delivery in living systems via a novel liposomal composition.

### BACKGROUND OF THE INVENTION

Efficient delivery of macromolecular drugs to remote tissues in the body is still considered a major unsolved problem in pharmaceutics. The rapid degradation and/or plasma clearance of these molecules affords little opportunity to reach remote tissues after parenteral administration, motivating the search for an advanced delivery vehicle for these drugs. Examples of such macromolecular drugs may include antisense oligonucleotides, short interfering RNAs and derivatives thereof, peptide nucleic acids, antibodies, hormones, insulin, enzymes, superoxide dismutase, polysaccharides, and heparin.

The immensely valuable new generation of nucleotide and peptide based therapeutics has in large part been unable to realize its clinical potential due to inherently short half lives caused by molecular weights below the renal clearance threshold as well as rapid degradation via enzymatic processes. For example, the half-life of a modified phosphothioate DNA oligonucleotide between 18 and 26 bases long is about 30-45 minutes in primates. Furthermore, like most other biomacromolecules, they must be administered parenterally due to instability and poor uptake from the digestive tract.

The past and present standards of parenteral delivery are subcutaneous (s.c.), intramuscular (i.m.), intraperitoneal (i.p.) and intravenous (i.v.) injections of a simple molecular solution of drug in a pharmaceutically acceptable carrier, typically in an aqueous media. These methods allow a fragile macromolecular drug to enter the body intact, but do nothing to alleviate the rapid clearance or inactivation of the drug during its distribution to disease targets. This necessitates inconvenient daily or weekly injection regimens, in certain cases taking 2 or more hours to complete each time, reducing patient compliance, increasing costs, and causing many promising macromolecular drug candidates to be abandoned or experience sub-optimal clinical outcomes. Alternative delivery methods for macromolecular drugs have been the subject of intense research; however, very few systems have reached advanced clinical testing or FDA-approval. Paragraphs 5-12 outline unsuccessful attempts at macromolecular delivery.

Transdermal delivery is being investigated as a parenteral route for smaller and more stable macromolecular drugs, such as insulin. Chemical excipients, acoustical or electrical fields, and microscopic arrays of needles have all been employed to increase stratum corneum permeability to macromolecules. However, the methods are inefficient and slow, they can be inconvenient and irritating causing poor patient compliance, the dose of macromolecule that can be passed across the skin is typically less than a few milligrams, and the method does nothing to protect or target the drug to disease sites once it enters the body.

Inhalation of a dry powder or nebulized liquid containing macromolecular drug has been explored to exploit the large and relatively permeable interior surface of the lung. However, degradation of the drug by the defensive mechanisms of the living lung, operational complexity leading to poor patient compliance, and the poor permeability of most macromolecules into the alveolar capillaries, remain major problems. Also, the method does nothing to protect or target the drug to disease sites once it enters the body if the target site is other than the lung.

Lactide/Glycolide-based polymer microparticles can controllably release peptides and nucleotides from a regional injection site (i.m. or s.c.) up to 30 days or more. However, all depot devices do nothing to improve the stability of the drug once in the circulation. Therefore, the distinct problem of directing the drug to a remote target, such as a tumor metastasis or infection site, remains. Also, the limited volume of most depots limits the total drug dose to 100 mg or less, making the technology only appropriate for relatively potent drugs.

Portable infusion pumps allow for a controlled and extended parenteral delivery of nearly any quantity of any drug, but are hampered by being bulky, uncomfortable, and subject to patient misuse and noncompliance. Furthermore, like polymeric depot injections, the device does nothing to stabilize or tissue target the fragile drug once it is released into the bloodstream.

Tethering hydrophilic polymers to a drug molecule, such as PEGylation, has succeeded at extending the pharmacokinetics of some proteins; however, this represents a covalent increase of the drug molecular weight, which is a direct inhibitor of membrane permeability and hence intracellular delivery. This greatly reduces the activity of a peptide or nucleotide-based drug, such as antisense oligonucleotides, siRNA, or peptide nucleic acids, whose site of action is intracellular.

Cationic liposome/polymer complexes have received virtually all of the focus in nucleoside delivery, due to electrostatic interaction with anionic cell membranes which greatly improves cellular internalization in vitro (Barron, 1999, and Ma, 2002). However, cationic particles form multi-micron aggregates in the anionic serum environment. Therefore, they have extremely short circulation kinetics and cannot achieve appreciable DNA levels in target tissues such as distal tumor sites (Mahato, 1995). The strong multivalent electrostatic interactions between DNA and cationic lipids or polymers, inherently lead to a carrier which does not release its DNA in a ready or predictable manner. Furthermore, cationic lipids and polymers present critical toxicity limitations, new chemical entities, and are clinically unproven in any capacity.

Neutral or anionic liposomes comprised of rigid high phase transition temperature lipids, above 40 degrees Celsius, with 20 to 60 mol % of cholesterol (Chol) have also been used to entrap and deliver macromolecular drugs (U.S. Pat. No. 6,333,314). These liposomes may be optionally sized to 150 nm or less to avoid rapid accumulation in the reticuloendothelial system (RES). Common example lipids include distearoyl phosphaticylcholine (DSPC), distearoyl phosphatidylglycerol (DSPG), distearoyl phosphatidylethanolamine (DSPE), and hydrogenated soy phosphatidylcholine. These compositions may further contain 1-10 mol % of a specialized lipid, or polymer-grafted lipid, for steric stabilization which leads to even further extended circulation times (Lorna, 2002). These steric stabilized liposomes are also known as stealth liposomes. However, while these highly stable lipid compositions, containing cholesterol and high transition temperature lipids maybe beneficial in the retention of small amphiphilic molecules, such as anthracyclines, vinca-alkaloids (Zhu, 2000), and fluoroquinolones (Irma, 2001), they can obstruct the timely release of macromolecular drugs. In this case, the ultimate disposition of the drug becomes that of the liposome itself: accumulation in the RES. For the fraction of liposomes that do reach the target site, the macromolecular drug simply remains entombed in an overly-stable liposome. The prevailing finding in liposome delivery of macromolecules is that despite improvement in pharmacokinetic parameters, existing conventional and stealth liposome designs result in efficacy generally no better than that of free drug. For example, ras antisense oligonucleotide (ISIS 2503) in dipalmitoyl phosphatidylcholine/Chol/PEG2000-DSPE stealth liposomes show no statistical improvement over free drug against tumor xenografts, despite dramatically improved pharmacokinetics (U.S. Pat. No. 6,083,923).

In summary, all of these parenteral devices/methods share critical shortfalls: (1) a lack of drug targeting (2) insufficient protection of the drug from degradation and clearance mechanisms in the bloodstream during distribution to target tissue or cells and/or (3) insufficient release of the macromolecular drug once at the target site.

### SUMMARY OF THE INVENTION

The present invention comprises liposomal delivery systems specifically designed for the delivery of macromolecules, including proteins, peptides, polysaccharides and oligonucleotides, to tumors, sites of inflammation or infection, or other disease sites possessing fenestrated vascular tissue. Lipid compositions of the liposome have been developed which yield dramatic and unexpected enhancements in the efficacy of delivery of macromolecules to cellular targets subsequent to intravenous administration. The liposomes of the invention protect the drug from potentially rapid enzymatic degradation and renal or RES clearance in vivo. The circulation half-life is shown to extend from about 30 minutes to about 10 hours. The invention lies within the class of 50-200 nm sterically-stabilized liposomes which entrap a drug and which are clinically proven to passively accumulate at disease sites, including tumors, infections, and inflammations. This liposome invention relates to specific phospholipid membrane compositions of sterically-stabilized liposomes directed at the delivery of macromolecular drugs.

The prevailing thought in the field of liposomal delivery has been that employing a liposome membrane of the greatest stability against drug leakage coupled with a PEGylated steric protective layer to ensure long circulation times, will result in optimal drug delivery efficacy (Charrois et. al., 2004 and Chou et. al., 2003). Cholesterol has historically been a key additive to help achieve this stability (U.S. Pat. Nos. 5,468,499, 5,814,335, 6,333,314). The ability of cholesterol to increase the cohesive strength and reduce the membrane permeability of phospholipids bilayers is well known (Needham and Nunn, 1990; Grit and Crommelin, 1993). There is also historical concern that intravenous injection of liposomes without cholesterol could contribute to erythrocyte fragility (Bruckdorfer et al, 1969). For these reason, drug delivery via sterically-stabilized liposomes has heretofore nearly exclusively employed high levels of cholesterol (20-60 mole %) in combination with high lipid chain melting temperature lipids or lipid mixtures. The melting temperature is typically above 45 degrees Celsius, as determined by differential scanning calorimetry. Without wishing to be bound to a particular theory, we suspect that poor past efficacy of sterically-stabilized liposomes containing water soluble macromolecules is attributable to these existing compositions being overly stable against release of their encapsulated therapeutic agents, thwarting bioavailability of the drug at the intended disease site (U.S. Pat. No. 6,083,923). It is the inherently slow permeability of macromolecules across these cholesterol-laden high chain melting temperature lipid bilayers in vivo, relative to small amphiphilic hydrophobic molecules, that necessitates a new specialized membrane composition allowing for release kinetics which optimize bioavailability at the target site(s).

The liposome designs of this invention specifically avoid high chain melting temperature lipid or lipid mixtures, avoid cationic lipids, and avoid cholesterol or sterols, in order to optimally release entrapped macromolecules in vivo. We hypothesize that for 50-200 nm diameter sterically-stabilized liposomes, similarly long intravenous circulation times are achieved for a wide variety of underlying membrane compositions—irrespective of anionic lipid charge, lipid chain melting temperature, degree of lipid unsaturation, and cholesterol content (FIG. 1). Hence, the underlying lipid membrane composition and its rate of degradation in vivo may be independently optimized in order to tune drug release rates, meanwhile maintaining the long circulation times and concomitant passive accumulation at certain disease sites, such as those possessing a leaky vasculature. In particular, a liposome comprising of low chain melting temperature lipids or lipid mixtures, below 45 degrees C., are specified in order to achieve sufficient release rates. These design rules achieve liposome carriers which are stable enough for shelf storage and the intravenous distribution phase (6-12 hrs), yet unstable enough to release the active drug rather than entombing it and rendering it inactive in vivo.

To these ends, molecular compositions for sterically-stabilized liposomes were developed which yield dramatic enhancements in the efficacious delivery of encapsulated macromolecular drugs to cellular targets after parenteral administration relative to prevailing sterically-stabilized liposome compositions. All compositions contain an arbitrary amount of encapsulated macromolecular drug which may exist in a dissolved, partially precipitated, or fully precipitated form. Precipitation may occur as a result of concentration, temperature, a pro-drug form of the macromolecule, or via pharmaceutically acceptable salts. Within the scope of this invention, the relevant drug is any hydrophilic macromolecule having a molecular mass greater than 3000 Daltons. A hydrophilic macromolecule typically possesses good intrinsic aqueous solubility, around 10 mg/ml or more, and a low octanol: water partition coefficient of less than 0.01. However, self-aggregation may limit solubility, for example with some proteins.

A preferred embodiment composition comprises 50-200 nm diameter unilamellar liposomes of 3-9 mole % of Polyethyleneglycol(2000)-Distearoylphosphatidylethanolamine (PEG2000-DSPE) and 97-91 mole % of Dipalmitoyl phosphatidylcholine (DPPC), which will be referred to as F1. A second composition comprises 50-200 nm diameter unilamellar liposomes of 3-9 mole % of Polyethyleneglycol(2000)-Distearoylphosphatidylethanolamine (PEG2000-DSPE) and 97-91 mole % of Dimyristoyl phosphatidylcholine (DMPC), which will be referred to as F4. A variety of other sterically-stabilized liposome compositions are possible that achieve similarly optimal release rates of hydrophilic macromolecules. The stability of a sterically stabilized liposome against drug leakage in blood is dominated by lipid chain length and degree of saturation. Hence, in the above examples, DPPC may be substituted with various proportions of dipalmitoyl phosphatidylglycerol (DPPG), dipalmitoyl phosphatidylethanolamine (DPPE), or dipalmitoyl phosphatidylserine (DPPS). Similarly, DMPC may be substituted with various proportions of dimyristoyl phosphatidylglycerol (DMPG), dimyristoyl phosphatidylethanolamine (DMPE), or dimyristoyl phosphatidylserine (DMPS). Synthetic derivatives of these lipids which possess saturated hydrocarbon chains of the same length are also incorporated herein. Furthermore, sterically-stabilization may be achieved by substituting PEG2000-DSPE with a variety of other specialized lipids (phosphatidylinostisol, ganglioside M1), polymer-derived lipids (PEG5000-DPPE, PEG-ceramides, etc), block copolymers (poloxamers), or other materials which result in enhanced circulation times relative to analogous liposomes lacking that material.

Other possible embodiments or modifications:
(1) Parenteral delivery of liposome entrapped macromolecular drugs or any other drug having very low lipid membrane permeability similar to that of a DNA oligonucleotide.
(2) Macromolecular drug may be encapsulated under conditions of concentration, temperature, salts, etc, which results in the drug being in a precipitated form, or a combination of dissolved and precipitated forms. The macromolecule may also exist in a form where multiple molecules are naturally associated, as in a tetrameric protein. This aggregate form may exceed 100 kDa in size.
(3) The macromolecular drug may be encapsulated in the liposome in combination with any stabilizers or cofactors necessary for the stability and biological activity of that drug. This would include protectant sugars, solubility enhancing surfactants, antioxidants, calcium, EDTA, etc.
(4) Administration by intravenous injection taking between several seconds or several hours, subcutaneous injection, intramuscular injection, intraperitoneal injection, inhaled deposition to the lung or nasal tissues, or topical administration to skin or eye.
(5) Delivery to target tissues of: blood, central nervous system, vital organs, primary and metastatic tumors, inflammation sites, rheumatoid arthritis, infection sites including: bacterial, fungal, or viral, and neovascularizing disease sites including: tumors, endometriosis, and macular degeneration.
(6) Treatment of diseases such as: cancer, endometriosis, macular degeneration, rheumatoid arthritis, bacterial infection, viral infection, psoriasis, or any condition benefiting from treatment via a macromolecular drug.
(7) Treatment of any disease where the liposome invention is combined with another treatment modality including: cancer chemotherapy, radiation, hyperthermia, or gene therapy antiviral, antifungal treatments, antibacterial treatments, anti-inflammatory drugs and treatments, drugs for rheumatoid arthritis, and treatments for the central nervous system. The amount and proportion of each agent may also be optimized to the condition of each particular patient.
(8) Attaching molecules to the inner or outer surface of the invention liposome which are designed to bind or interact with a specific target in the body. Examples include a protein which interacts with or binds to a specific cellular target molecule, thereby anchoring the liposome in place, inducing cellular endocytosis of the liposome, or inducing a response in the liposome itself.
(9) Any method of making the liposomes of the invention known to those skilled in the art, including those associated with: microemulsification, microfluidization, extrusion through filters of a defined pore size, hydration of a lipid film, repeated freeze-thaw, solvent injection, sonication of a lipid dispersion, freeze-drying/lyophilization followed by rehydration, reverse phase evaporation, etc.
(10) It may be necessary to lyophilize, freeze-dry, or otherwise create a dry powder form from a liposome suspension, using established protectants like high concentrations of certain sugars, followed by reconstitution in a pharmaceutically acceptable aqueous carrier. Alternatively, the liposome suspension may be stored by being directly frozen from a liquid state, at freezing rates ranging from slow to vitrification. Various cryoprotectants sugars like sucrose, or trehalose, can be added. The liposome suspension can be maintained in frozen state until thawed for use, being thawed at slow or fast rates at a temperature not exceeding the main lipid chain melting temperature of the liposome membrane.

### BRIEF DESCRIPTION OF THE DRAWINGS

FIG. 1: is a schematic of the liposomal delivery vehicle. The liposome is between 50-200 nm in diameter and sequesters the macromolecular drug in the interior of the phospholipids bilayer shell. The phospholipids bilayer can be comprised of any mixture of vesicle-forming lipids resulting in a lipid chain melting transition temperature of between 10 and 45 degrees Celsius. The liposome can be further doped with a polymer-lipid which acts as a steric stabilizer.

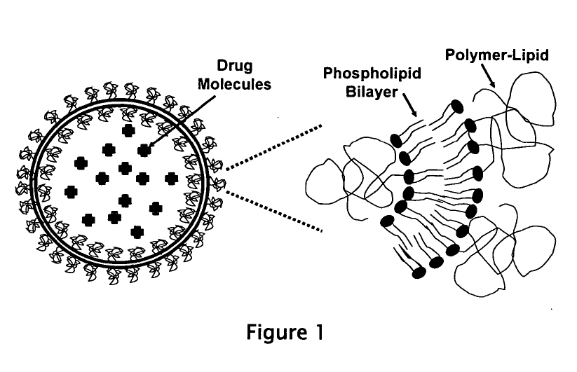

FIG. 2: shows the release of an oligonucleotide from formulation F1. The release study was performed at 37 degrees Celsius in a saline medium. The lipid bilayer of formulation F1 is in the pretransitional state at this temperature.

FIG. 3: shows the tumor growth curves of mesothelioma xenografts in nude mice treated with VAS formulations. The nude mice were injected with formulations at days 0 and 12. F1 is seen to effectively suppress tumor growth. After the second treatment on day 12, one of the F1 treatment groups resumed tumor growth while the other remained in remission. Both F2 groups showed steady tumor growth before and after treatment.

FIG. 4: shows the reduction in final tumor weights achieved via treatment with VAS-bearing F1 relative to control.

FIG. 5: shows tumor growth curves of mesothelioma xenografts in nude mice treated with VAS formulations. Tumor growth was compared between untreated mice (control) and mice injected weekly with free VAS (vegfas 3) or F1 (F1-1), and mice injected twice weekly with formulation F1 (F1-2). Treatment using the Invention showed significant reduction in tumor growth over the entire study.

FIG. 6: Compares tumor growth curves of mesothelioma xenografts in nude mice with varying liposomal VAS formulations. Mice were treated with weekly injections (twice weekly in the case of F1-2). Inventions F1 and F4 performed significantly better at inhibiting tumor growth over conventional formulations.

FIG. 7: Fluorescent micrographs of mesothelioma xenograft tumor growth in nude mice. The endothelial cells are stained by fluorescein-lectin after sacrifice on the 21^{st} day. Control mice were left untreated, whereas F1-1 mice were treated with F1 VAS formulations on a weekly basis. The F1 treated mice were observed to have down-regulated VEGF expression and dramatically reduced endothelial cell proliferation.

### DESCRIPTION OF THE PREFERRED EMBODIMENTS

The liposomes of this invention can be produced by any of the various methods known to one skilled in the art, including combinations of lipid film hydration, repeated freeze-thaw, extrusion through filters or a desired pore size, reverse phase evaporation, probe or bath sonication of lipid dispersion, solvent injection, dehydration-rehydration, interdigitation-fusion, etc. There were five liposomal compositions prepared in our studies for analysis and comparison. The formulations are outlined in Table 1:

TABLE 1
Lipid compositions for the delivery of oligonucleotides

| | | | | Lipid:Drug |
|---|---|---|---|---|
| | | | | Mass |
| Name | Lipid composition | Mole Ratio | Size (nm) | Ratio |
| F1 | PEG 2000-DSPE:DPPC | 5:95 | 101-123 | 20 |
| F2 | PEG 2000-DSPE:EggPC | 5:95 | 112 | 20 |
| F3 | PEG 2000- | 5:55:40 | 107-119 | 20 |
| | DSPE:DPPC:Cholesterol | | | |
| F4 | PEG 2000-DSPE:DMPC | 5:95 | 101 | 20 |
| F5 | PEG 2000- | 5:55:40 | 110-128 | 20 |
| | DSPE:DSPC:Cholesterol | | | |

The following examples are provided to illustrate the present invention and should not be construed to limit the scope of the invention of the present application, as defined in the claims which follow thereafter, in any way.

### EXAMPLE 1

### Liposome Formulation and Stability Testing

Liposome formulation F1 was constructed by weighing 83.2 mg of DPPC and 16.75 mg of PEG2000-DSPE (equivalent to 5 mole %) into a 10 ml glass vial. 100 microliters of ethanol were added and the mixture was heated to 50 degrees C. to dissolve the lipids. The solution was then cast into a film on the interior of the glass vial, and the residual ethanol was removed under vacuum overnight at 20 degrees C. The lipid film was then hydrated with 1 ml of a 5 mg/ml phosphothioate oligonucletide solution (21 bases) in DNAse-free isotonic saline. The mixture was heated to 60 degrees C. with mild vortexing for 1 hour to completely suspend the lipid film. A milky white lipid suspension results, with large heterogeneous multilamellar vesicles being formed. Then the mixture was alternately frozen and thawed 16 times by alternating the vial between a liquid nitrogen bath and a water bath at 50 degrees C., with gentle stirring. This produces a more fluid and translucent suspension of smaller liposomes. The liposomes encapsulating the oligonucleotide were then extruded 11 times through polycarbonate filters bearing well-defined 100 nm pore sizes, at 55 degrees C. Liposome size was verified to be 112 nm via photon correlation spectroscopy and analyzing data by the method of cumulants.

The 1 ml solution was then diluted with 2 ml of isotonic saline to a final lipid concentration of 33 mg/ml and an oligonucleotide concentration of 1.7 mg/ml. An aliquot was taken, and the fraction of encapsulated drug was determined by a combination of filtration and spectrophotometric assay to be 25%. Unencapsulated oligonucleotide was removed from the formulation by dialysis in a 300,000 MWCO membrane against isotonic saline for 24 hrs. A 40 ml volume was exchanged twice, ensuring less than 1% of remaining free drug. This resulted in a purified F1 formulation containing about 0.65 mg/ml oligonucleotide.

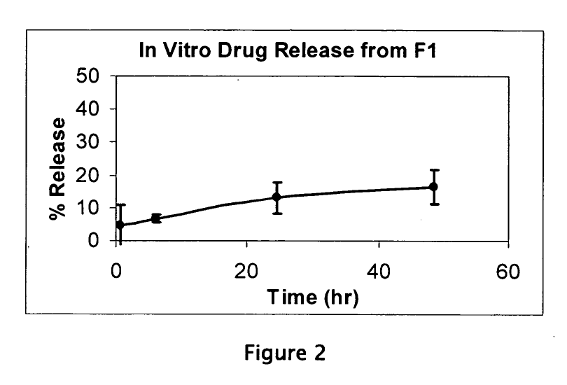

To measure the in vitro macromolecular release of formulation F1, one milliliter of the purified F1 liposomes, containing encapsulated oligonucleotide, was diluted with 10 ml of DNAse-free isotonic saline and maintained at 37 degrees C. with slow stirring. At several time points, a 2 ml sample was removed and assayed for released oligonucleotide via a combination of filtration and spectrophotometric assay. Data was reported as the average of two assays (FIG. 2). A steady slow release of oligonucleotide from F1 is observed. At 37 degrees C., DPPC is in the “pretransition state” of the lipid chain melting transition, and so the release mechanism may be lipid reorganizations and packing defects known to increase permeability.

Short-term storage stability of formulation F1 was also assessed. A sample of F1 was stored at 4 degrees C. After 11 days, leakage of drug was below detection limits, equating to less than 5% leakage. This is possible due to the lipid chain melting temperature of F1 being about 42 degrees C., resulting in a solid-chain state at 4 degrees C. which is resistant to leakage of large molecules. On the other hand, the F2 formulation (Table 1) showed an unacceptable 15% leakage from the liposomes during the same time. The unsaturated lipids in F2 result in a fluid membrane with a transition temperature below 4 degrees C. that is more likely to allow leakage via lipid reorganizations, such as transient pore formation.

### EXAMPLE 2

### Preliminary in Vivo Efficacy Test

The model macromolecular drug selected for in vivo testing was a proprietary VEGF antisense oligonucleotide referred to as “VAS”. By suppressing cellular expression of VEGF, VAS suppresses the biological signals that are integral to most angiogenic disease processes as well as autocrine/paracrine growth of certain tumors cells. VAS is proven to be effective at suppressing VEGF expression in animals, but suffers from a very short (30 min) plasma half-life in vivo, requiring extended daily intravenous infusions for optimal efficacy. Furthermore, plasma clearance is primarily via the kidneys, leading to renal toxicity as the dose limiting toxicity. Our liposome formulation possesses the potential to greatly improve pharmacokinetics and tissue targeting, and reduce toxic exposure to vital organs, for oligonucleotides and other macromolecular drugs which are rapidly metabolized or cleared from the blood.

Human mesothelioma is an ideal model of the cellular VEGF-mediated proliferation and neovascularization that is associated with it and many other diseases, including most cancers, endometriosis, wet-type macular degeneration, and even some inflammatory conditions. Mesothelioma proliferation and neovascularization is strongly dependent upon VEGF expression. A reduction in the growth of VEGF responsive tumors and associated vascular tissue, relative to untreated controls, would indicate effective downregulation of VEGF, and hence effective cellular delivery of the antisense oligonucleotide by our liposome vehicle.

In general, optimizing a delivery system is best done using in vivo efficacy as the ultimate metric in cases where the pharmacokinetics of the drug and the liposomal carrier are well-understood. In contrast, others have based conclusions about the optimal or effective liposomal compositions for delivery of oligonucleotides and other macromolecular drugs largely on pharmacokinetic and distribution data. Unfortunately, these assays typically do not distinguish between the encapsulated and free, biologically active, forms of the drug. Thus an overly-stable liposome formulation may display misleading improvements in pharmacokinetics and tumor accumulation for a macromolecular drug, yet exert little if any biological activity because the drug is not being properly released due to a combination of high transition temperature lipids and cholesterol. For example, ras antisense oligonucleotide (ISIS 2503) in DPPC/Chol/PEG2000-DSPE stealth liposomes show no statistical improvement over free drug against tumor xenografts (U.S. Pat. No. 6,083,923), despite dramatically improved pharmacokinetics. Hence, without understanding the source of the problem, studies have concluded that liposomes do not work and have abandoned them for hydrophilic macromolecules.

To conclusively demonstrate the detriments of excessive liposome stability to in vivo efficacy, a library of liposome compositions spanning a broad range of stability was constructed (Table 1). For comparability, all compositions were close to 100 nm in size and comprised only saturated, neutral lipids, identical PEG-lipid coatings, and the same amount of encapsulated VAS. For all formulations, VAS was encapsulated using lipid hydration followed by freeze-thawing and extrusion through 100 nm polycarbonate filters. The resulting liposome size was verified by photon correlation spectroscopy, with free drug removed via dialysis against isotonic saline, to a final VAS concentration of about 2 mg/ml.

It is known that increasing lipid chain length, degree of saturation, and cholesterol content all contribute to greater relative liposome stability against leakage of encapsulated contents in vivo. Approximately speaking, the relative rank of the test formulations in Table 1 would thusly be, in order of increasing stability: F4<F2<F1<F3<F5. The historical clinical development of liposomal delivery was focused on hydrophobic/amphipathic chemotherapeutics and antibiotics, for which liposome stability was considered universally insufficient, prompting the focus on highly stable lipid and cholesterol compositions such as F3, F5, and others including hydrogenated soy phosphatidylcholine and cholesterol (U.S. Pat. Nos. 5,468,499, 5,814,335, 6,333,314).

In a distal tumor xenograft model, liposomes which potentially lack cholesterol and high phase transition temperature lipids must possess a steric-stabilization layer in order to sufficiently circulate and accumulate in tumors. Likewise, a 100 nm liposome size was finalized because it has been previously determined that the optimal diameter for accumulation in tumor xenografts after intravenous injection is between 80-150 nm (Charrois and Allen, 2003). Cationic lipids or polymers complexed with antisense oligonucleotides are known to be effective at intracellular delivery in vitro, but also show inferior circulation half-life and tumor accumulation after intravenous administration (U.S. Pat. No. 6,333,314). Hence, the library of compositions tested are reasonably expected to be the best alternatives presently known to the art of lipid drug carriers with respect to intravenous delivery of macromolecular drugs to distal disease sites possessing a leaky vasculature.

F1 liposomes having a size of 100 nm, and containing VAS encapsulated at a 20:1 lipid to VAS weight ratio, were injected into nude mice bearing human mesothelioma xenografts formed by injecting 6.5 million cells subcutaneously on day zero. Mice received fast bolus tail vein injections of F1 of approximately 10 mg/kg body weight of VAS on days 2 and 12. As a comparison, F2 (see Table 1) was given in the same manner on day 12.

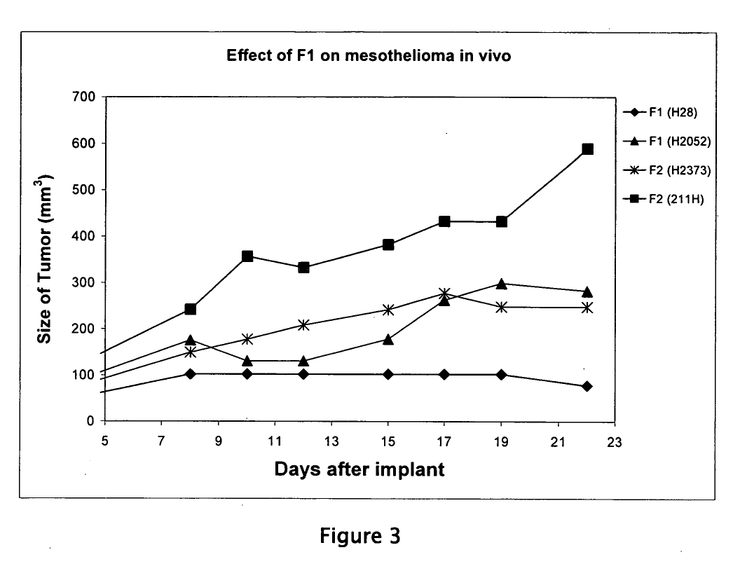

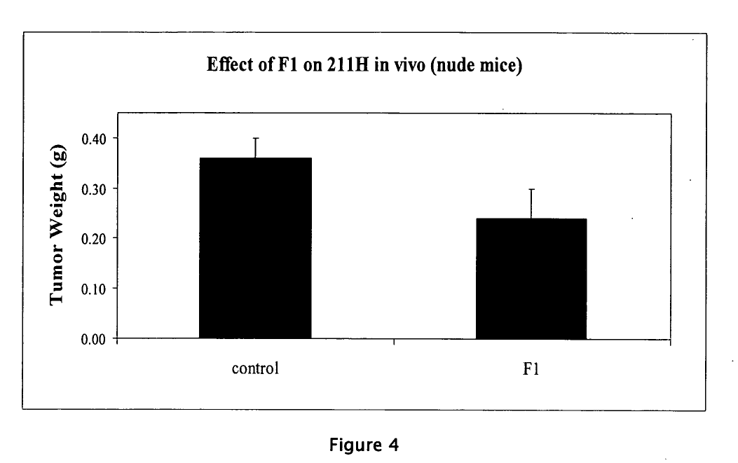

Tumor growth curves in FIG. 3 show that F1 effectively suppresses tumor growth. After the second treatment on day 12, one of the F1 treatment groups resumed tumor growth while the other remained in remission. Both F2 groups showed steady tumor growth before and after treatment. FIG. 4 shows the reduction in final tumor weights achieved via treatment with VAS-bearing F1, relative to control.

### EXAMPLE 3

### In vivo Testing of Liposome Library

In order to more clearly assess the novelty and efficacy of the invention, the library of liposomal VAS compositions (Table 1) was compared in the human mesothelioma tumor xenograft model. Approximately 5 million mesothelioma tumor cells were injected subcutaneously into each nude mouse on day zero. Five mice were used in each group to assure good statistical significance of results. Starting on day one, liposomal formulas were given once per week via rapid tail vein bolus injection of approx 10 mg/kg. F1 was also given to a separate group as two injections per week (F1-2, whereas F1-1 is F1 given once weekly). As a control, one group was treated with naked oligonucleotide in saline, and an untreated group was maintained as a further control. The number of mice in each treatment group was five. After 21 days of treatment, the mice were sacrificed.

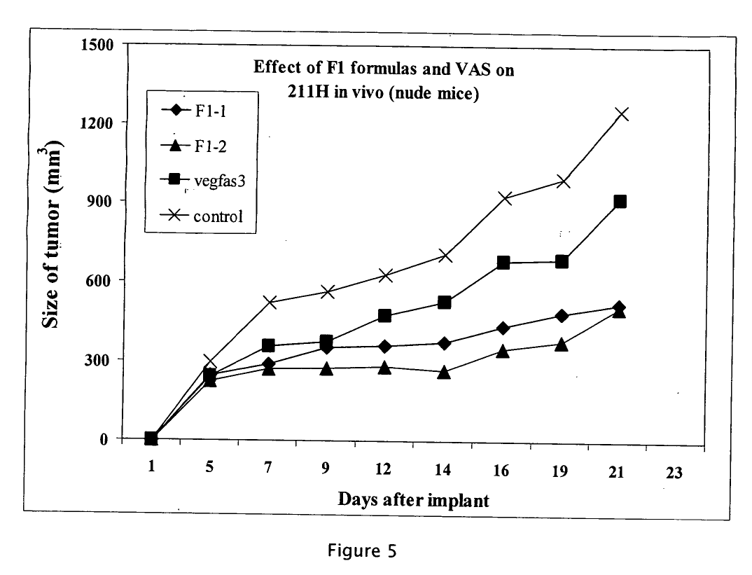

The resulting tumor growth curves in FIG. 5 show a dramatic result. The Invention, F1-1, is again very effective at suppressing VEGF-dependent xenograft growth, relative to untreated controls, and treatment with free drug. Administration of F1 twice weekly (F1-2), further improves performance, demonstrating a dose-dependent response.

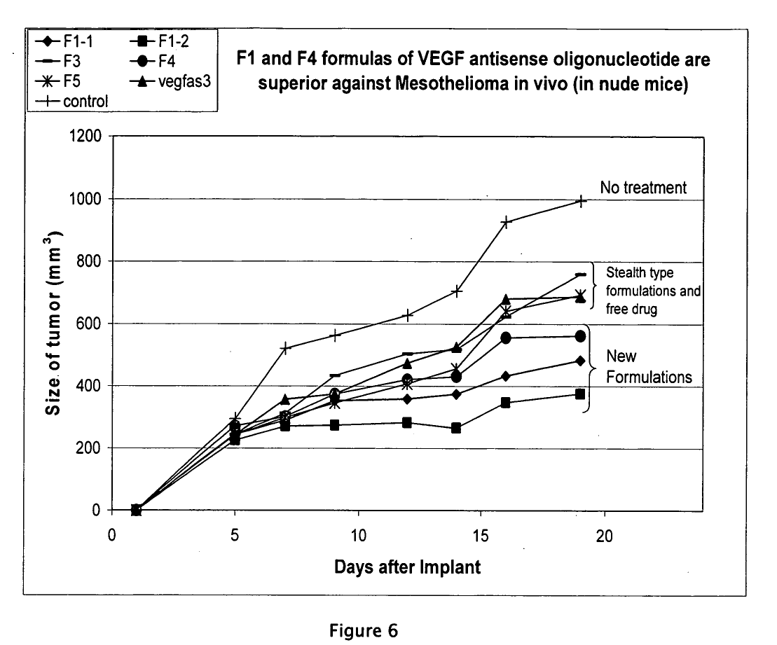

F1 proved to be significantly superior to all other tested liposome compositions, the two most stable of which (F3 and F5) performed no better than an equivalent injection of free drug (FIG. 6), as one might predict based on prior macromolecule delivery studies involving high phase transitions temperature lipids (>40 degrees C.) combined with greater than 20 mol % cholesterol. F4 is also considered successful and novel relative to existing stealth liposomes (F3 and F5). F4 contains no cholesterol, and importantly, has a low transition temperature lipid (DMPC transition temperature is equal to 23 degrees C.) creating a fluid lipid membrane at physiological temperature, and thus an increased rate of release of macromolecules relative to solid lipid membranes (such as F3 and F5).

To our knowledge, this is the first side-by-side test of this kind conducted with a macromolecular drug encapsulated in a library of neutral stealth liposomes. The identification and satisfaction of a sensitive requirement for the in vivo membrane stability and hence rate of macromolecular drug release from these liposomes has not before been reported. The significance and novelty of the most effective compositions are underscored by the great chemical and physical similarity between all liposome compositions tested: all are neutral, 100 nm, saturated-chain, unilamellar liposomes coated with a 5 mol % PEG2000-DSPE layer. The circulation time, accumulation at the target/disease site, and rate of cellular internalization of all tested liposomes are thus similar, thereby suggesting the only significant difference between them is the rate of drug release. Without wishing to be bound to any particular theory, this surprising new efficacy is achieved through designing liposome compositions having an effective in vivo release rate of macromolecule. Compositions F3 and F5 are likely too stable, suggesting the macromolecular drug is not liberated from the liposome before the bulk of the liposomes are cleared from the bloodstream. Liposomes which do reach the target site might never release the drug by virtue of excessive stability. It could also mean that endocytic lysosomal degradation of the liposome and its entrapped drug occurs before the drug can be released. Composition F2 is probably too unstable, releasing too much macromolecular drug before there is sufficient time for the liposomes to accumulate at the target site—resulting in efficacy not significantly different from a slow infusion of non-liposomal drug. It may also be that the less stable liposome formulations are opsonized and cleared from the bloodstream faster than the other compositions.

Barron, Uyechi and Szoka (1999), report that cationic lipids are essential for intracellularization and activity of plasmid DNA after intravenous injection in a lipid carrier. VAS biological activity also requires intracellular localization. Unexpectedly, though lacking cationic lipids, the liposomes of the present invention deliver VAS with high biological activity (FIGS. 2-6), refuting this report and further demonstrating the utility of the present invention, as cationic lipids display inferior pharmacokinetics and distribution in vivo (U.S. Pat. No. 6,333,314).

The novelty is again emphasized when one considers that the only difference between F3, which is less effective than free drug, and F1 is that F3 additionally contains 40 mol % cholesterol. Cholesterol has historically been viewed in the art of liposomal drug delivery as essential: it is contained in all clinically-approved intravenous liposome products, and persists in those in development. The prevailing belief is that cholesterol-free compositions such as F1 are inferior to more stable cholesterol containing compositions (F3 and F5) with regards to intravenous drug delivery and tumor targeting (U.S. Pat. Nos. 5,468,499, 5,814,335, 6,333,314). Our contradicting experimental findings dispute this concept, and suggest that cholesterol can lead to overly stable liposomes which thwart the optimal release and bioavailability of a macromolecular drug at a disease site.

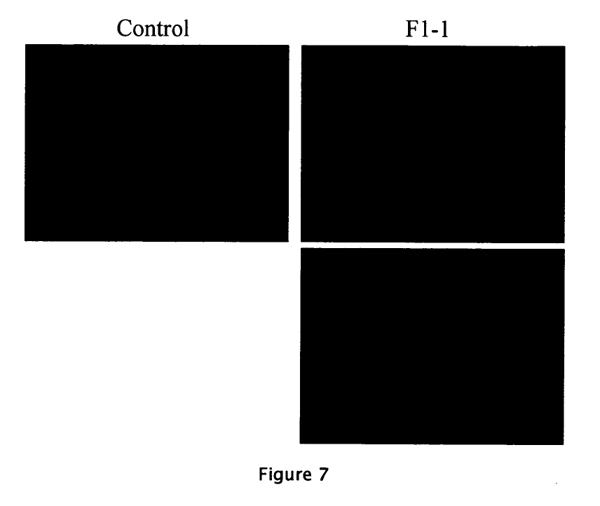

The above conclusions, based on xenograft growth curves, are corroborated with fluorescent microscopy of thin sections of the tumor mass. Endothelial cells were stained by fluorescein-lectin after sacrifice on day 21 (FIG. 7). Once per week administration of VAS via F1-1 is observed to down-regulate VEGF expression and dramatically reduce endothelial cell proliferation associated with angiogenesis.

In conclusion, experiments with liposomal VAS in the treatment of human mesothelioma xenografts show that novel drug activity can be achieved via avoidance of a lipid membrane phase transition temperature above 45 degrees C., avoidance of cholesterol or other membrane stabilizing sterols, and avoidance of cationic lipids. This goes against the teaching of decades of prior art in the liposomal drug delivery field (U.S. Pat. Nos. 5,468,499, 5,814,335, 6,333,314, and 6,534,484). However, the VAS data herein is simply an example of novel in vivo delivery of the broad class of macromolecules using a specialized liposomal composition, and is not meant to limit the scope. Within this Invention, other types of macromolecular drugs could be delivered to treat disease sites of angiogenesis, infection, or inflammation.

1. A liposome composition for delivery of a therapeutic agent, comprising:
(a) From 1 to 10 mol percent of one or more lipids derivatized with a hydrophilic polymer
(b) the remainder of the lipids not containing cholesterol, sterols or derivatives thereof and not bearing a positive net charge when between pH 5 and 9, wherein said lipids possess a measurable gel to fluid lipid membrane phase transition temperature that exists between 10 and 45 degrees Celsius when each unique lipid component is measured individually in the fully-hydrated state between pH 5 and 9.
(c) a therapeutic or diagnostic agent, having a molecular weight greater than 3000 Daltons, that is completely encapsulated inside the liposome.

2. The composition of claim 1, wherein the therapeutic agent comprises a peptide, nucleotide or polysaccharide of a naturally occurring or chemically modified molecular structure.

3. The liposome of claims 1 and 2, wherein one or more lipids are selected from the group consisting of: phosphatidylcholine, phosphatidylglycerol, phosphatidylserine, phosphatidylethanolamine, or sphingomyelin.

4. The liposome of claims 1-3 consisting of a size between 30 and 1500 nm.

5. The liposome of any one of claims 1-4, wherein the acyl chains are selected from the group consisting of: myristoyl (14 carbons per chain), or palmitoyl (16 carbons per chain).

6. The liposome of claim 1-5 wherein said lipid derivatized polymer is a phosphatidylethanolamine derivatized with polyethylene glycol.

7. The liposome of claim 1-6 wherein said lipid derivatized polymer is a lipid conjugated hydrophilic polymer that extends circulation time in blood at least two fold over a comparable liposome of composition and size which lacks said lipid derivatized polymer.

8. A liposome composition for delivery of a therapeutic agent, comprising:
(a) From 1 to 10 mole percent of one or more lipids derivatized with a hydrophilic polymer
(b) the remainder of the lipids not containing cholesterol, sterols or derivatives thereof, and not bearing a positive net charge when between pH 5 and 9, wherein said lipids as a mixture have a measurable gel to fluid lipid membrane phase transition temperature between 10 and 45 degrees Celsius when the lipid mixture is measured in the fully-hydrated state between pH 5 and 9.
(c) a therapeutic or diagnostic agent, having a molecular weight greater than 3000 Daltons, that is completely encapsulated inside the liposome.

9. The composition of claim 8, wherein the therapeutic agent is a peptide, nucleotide or polysaccharide of a naturally occurring or chemically modified molecular structure.

10. The liposome of claims 8 and 9, wherein one or more lipids are selected from the group consisting of: phosphatidylcholine, phosphatidylglycerol, phosphatidylserine, phosphatidylethanolamine, or sphingomyelin.

11. The liposome of claims 8-10 consisting of a size between 30 and 1500 nm.

12. The liposome of any one of claims 8-11, wherein the acyl chains are selected from the group consisting of: myristoyl (14 carbons per chain), palmitoyl (16 carbons per chain).

13. The liposome of claim 8-12 wherein said lipid derivatized polymer is a phosphatidylethanolamine derivatized with polyethylene glycol.

14. The liposome of claim 8-13 wherein said lipid derivatized polymer is a lipid conjugated hydrophilic polymer that extends circulation time in blood at least two fold over a comparable liposome of composition and size which lacks said lipid derivatized polymer.
